(19) United States
(12) Patent Application Publication
(10) Pub. No.: US 2012/0189599 A1
(43) Pub. Date: Jul. 26, 2012

(54) MEDICATION FOR ORAL ADMINISTRATION, COMPRISING AT LEAST ONE ESTROGEN AND/OR AT LEAST ONE GESTAGEN AND AT LEAST ONE PROBIOTIC BACTERIAL STRAIN

(75) Inventors: Stefanie Lindemann, Berlin (DE); Sascha General, Berlin (DE)

(73) Assignee: BAYER PHARMA AKTIENGESELLSCHAFT, Berlin (DE)

(21) Appl. No.: 13/322,131

(22) PCT Filed: May 14, 2010
(86) PCT No.: PCT/EP2010/002965

§ 371 Date: Apr. 12, 2012

(30) Foreign Application Priority Data
May 22, 2009 (DE) 102009022947.7
May 27, 2009 (DE) 102009023632.5

(51) Int. Cl. A61K 35/74; A61P 15/02; A61P 5/34; A61P 13/00; A61P 5/30; A61P 15/18; A61P 13/10

(52) U.S. Cl. 424/93.45; 424/93.4

(57) ABSTRACT

The present invention relates to a medicament for oral administration containing at least one estrogen and/or one gestagen and at least one probiotic bacterial strain, such as for example lactobacillus species.

The medicament according to the invention is used either for oral contraception or for hormone therapy (HT), during which it can simultaneously serve for the stabilization of the vaginal environment and hence the prevention of infectious diseases, such as for example vaginal mycosis, bacterial vaginosis and/or bladder inflammation (bacterial cystitis) or the prevention of urogenital symptoms, e.g. dyspareunia and dysuria.

The present invention further relates to pharmaceutical combination preparations which contain dosage units containing an aforesaid medicament and further dosage units containing exclusively a probiotic bacterial strain.

The present invention relates to a medicament, which contains at least one estrogen and/or at least one gestagen and at least one probiotic bacterial strain (e.g. a lactobacillus).

The medicament according to the invention is configured such that it can be used either for oral contraception or for hormone therapy (HT) and thus at the same time can be used for stabilization of the vaginal environment and hence the prevention of infectious diseases, such as for example vaginal mycosis, bacterial vaginosis and/or bladder inflammation (bacterial cystitis) or the prevention of urogenital symptoms, e.g. dyspareunia and dysuria.

The pharmaceutical firms active in the field of fertility control are constantly endeavoring to improve the existing contraceptives. This includes not only increasing contraceptive reliability by development of new substances and improved comfort of use. Rather they are also pursuing innovative approaches to the combination of contraception and disease prevention.

In premenopausal women, the vaginal environment is shifted towards the neutral to basic by sexual activity, owing to the pH of the ejaculate. This has the consequence that the vaginal flora only capable of existence in the acidic range, e.g. the naturally occurring lactobacilli, is suppressed or replaced by urinary pathogens growing in the basic range (Candida, E. coli, A. vaginae or G. vaginalis).

As a result, for sexually active women there is an increased risk of contracting the aforesaid urogenital tract infections or symptoms. Admittedly, current standard therapies (metronidazole, clindamycin and antimycotics, etc.) enable substantial eradication of the pathogenic microbial flora; however because of their mechanism of action they are not capable of restoring the natural vaginal environment, including lactobacillus colonization (Marellli).

Associated with this is an increased risk of reinfection resulting in chronification. In addition, because of the repeated treatments, there is an increased risk of the development of a microbial flora largely resistant to standard therapies (Cribby; Hay).

The symptoms associated with these infections lead to considerable psychological stress in the women affected resulting in frequent medical consultations and/or inadequate self-medication. Depending on the colonization status as regards the probiotic bacterial species (rectal or intravaginal), age, race/ethnic origin, education level and social status of the woman, there is a high incidence of urogenital tract infection, e.g. bacterial vaginosis (Johannsen).

Literature statements on the incidence of bacterial vaginosis vary from 4 to 60% depending on the population studied. In the USA, for example, up to one third of all sexually mature women contract bacterial vaginosis (Allsworth).

70 to 75% of all women contract vulvovaginal vaginosis at least once in their life: 40 to 50% of all women contract it several times (Sobel).

Because of their high incidence and their high relapse rate, the urogenital tract infections represent a considerable burden on the budget available for health care. Additional costs to the state and to society arise through the losses of working hours caused thereby.

Urogenital tract infections, such as for example bacterial vaginosis, are a risk factor for premature births or are associated with an increased risk of the woman giving birth prematurely (Nelson).

In perimeno- and postmenopausal women, owing to the estrogen deficiency, the natural, lactobacillus-containing vaginal flora is suppressed by uropathogenic microbes and thus the vaginal environment is destabilized.

In peri- and postmenopausal women, the estrogen deficiency leads to a reduction in the supply of glycogen-positive vaginal epithelium and associated therewith to a reduction in the naturally occurring lactobacilli. As a result, this leads to a destabilization of the vaginal environment associated with a shift in the vaginal pH. These changes in the vaginal environment have the same consequences (pathogenic microbial invasion) as already described for premenopausal women.

The terms pre-, peri- and postmenopausal are utilized in the context of the present invention in the manner familiar to the person skilled in the art.

As women's age increases, the risk factors, such as for example age-related anatomical changes, immunological factors and/or reduced perfusion, also increase. Associated with this there is increasing incidence and chronification with increasing age. In particular, the treatment of older, often also multimorbid female patients necessitates a systematic treatment of urogenital tract infections. In this patient clientele, often under polypharmacological treatment, the risk of undesired drug interactions also increases with each additional therapy.

After the treatment of genital tract infections already described above, as a rule there follows the administration of lactobacillus-containing vaginal tablets or capsules for restoration of the healthy vaginal environment.

Here in some preparations a small addition of estrogens, for example estriol in the medicament Gynoflor®, is given to increase glycogen release and associated therewith to provide a further nutritional basis for the lactobacilli.

The positive effects of lactobacillus administration in patients suffering from bacterial vaginosis (Anukam; May) or from urogenital tract infections (Falagas; Reid a)) have been described many times in recent years.

The currently available, exclusively vaginal, presentations of lactobacilli do not allow the continuation of the treatment during the vulnerable menstruation phase. Likewise, the vaginal application of tablets and suppositories leads to undesired, compliance-inhibiting effects, for example the outflow of formulation residues, and to stinging, itching and redness.

It has however already been described that by means of oral administration of certain probiotic strains (Lactobacillus rhamnosus GR-1, Lactobacillus reuterii RC-14) it is possible to restore the normal vaginal flora in postmenopausal women (Petricevic).

It had already previously been shown that lactobacilli (Lactobacillus rhamnosus, Lactobacillus fermentum) can reach the vaginal region after oral administration (Marelli; Reid b)).

The adhesion of the lactobacilli as a function of the menstrual cycle (and hence as a function of the particular hormone status) has already been demonstrated in ex vivo/in vitro studies (Chan).

The connection between a low estrogen level and reduced lactobacillus colonization can also be observed in postmenopausal women (Falagas).

The present invention is based on the objective of creating a contraceptive or an HT preparation which minimizes the diseases described or the disease risks due to sexual activity in the aforesaid patient groups.

The invention is also based on the objective of discovering a medicament or treatment regime in the form of a pharmaceutical composition (kit), which ensures that the user of the medicament or the pharmaceutical composition according to the invention is also still reliably protected against urogenital tract infections for a further period after discontinuation.

According to the invention, a combination preparation is proposed, which is suitable for simultaneous oral use of the contraceptive or HT preparation and of the probiotic bacterial strain.

Hormonal, oral contraceptives in every case contain a gestagen (so-called POPs, progesterone only pill); however in most cases they contain an estrogen (in most cases this is ethinylestradiol) and a gestagen. Here, different administration and dosage regimes are known.

An HT preparation in every case contains an estrogen (preferably this is estradiol or estradiol valerate; however, ethinylestradiol is also possible) and in most cases also a gestagen. Here too, different administration and dosage regimes are known.

Hence one embodiment of the present invention relates to a medicament for oral administration containing at least one estrogen and/or one gestagen and at least one probiotic bacterial strain.

Further embodiments are stated in the dependent claims 2 to 14.

A further embodiment consists of a multiphase pharmaceutical combination preparation (kit) containing at least 20 daily dosage units containing a medicament for oral administration containing at least one estrogen and/or one gestagen and at least one probiotic bacterial strain and at least one daily dosage unit containing at least one probiotic bacterial strain, wherein the number of all dosage units contained in the kit is at least 28 and the dosage units are arranged such that first the dosage units containing the medicament for oral administration containing at least one estrogen and/or one gestagen and at least one probiotic bacterial strain and then the dosage units containing only the probiotic bacterial strain are to be taken.

Concerning further embodiments for the pharmaceutical combination preparation according to the invention, reference is made to the dependent claims 16 to 22.

If it is an oral contraceptive, the pharmaceutical combination preparation according to the invention is in particular administered as a 21+7 or as a 24+4 regime, i.e. 21 or 24 daily dosage units containing an estrogen and gestagen and a probiotic bacterial strain and 7 or 4 daily dosage units containing exclusively a probiotic bacterial strain.

These two regimes are represented diagrammatically in the following two figures I) and II):

Further, it is possible to use the medicament according to the invention in an extended administration cycle (“extended regimen”) or in a flexible administration cycle.

Only the oral administration of the probiotic bacterial strain in a pharmaceutical combination preparation for contraception enables the full expression of the already mentioned synergistic effects of the estrogen on the stabilization of the vaginal environment and the treatment with the lactobacilli in the vulnerable phase of menstruation.

In postmenopausal women, the advantage consists in the fact that on account of the oral administration route the treatment can be continued without restriction in spite of the symptoms attendant on dyspareunia and dysuria.

In every case through the administration of the combination of oral contraceptive or orally administered HT preparation and the probiotic bacterial strain, a continuous presentation of the probiotic bacterial strain is achieved. Because of the simultaneous ingestion with the contraceptive or the HT preparation, compliance is increased. As a result of this, women in a risk group (sexually active women or peri- and postmenopausal women) are treated continuously with probiotic bacterial strains and, attendant on this, with stabilization/restoration of the healthy vaginal environment. As a result, improved and continuous protection against urogenital tract infections and symptoms is achieved.

Through the administration of the medicament or the pharmaceutical combination preparation according to the invention, stabilization of the vaginal pH (3.5-4.2) over several weeks to several months after discontinuation of ingestion of the medicament or the pharmaceutical combination preparation is achieved (specialist information on Gynoflor®). This solves the initially posed problem of still reliably protecting the user against urogenital tract infections with the medicament or the pharmaceutical combination preparation for a certain time after discontinuation.

The ascent of uropathogenic microbes from the vaginal mucosa and the cervical inflammation environment developing therefrom, particularly in the first trimester, is a known risk factor for pregnant women to give birth prematurely (Simhan). In particular with the early onset of a pregnancy after ceasing taking the medicament, the user is thereby still protected against urogenital tract infections for a certain time. As a result, an important risk factor for giving birth prematurely is excluded.

As gestagens, for example the following substances can be used in the medicament according to the invention or in the pharmaceutical combination preparation according to the invention: levonorgestrel, norgestimate, norethisterone, dydrogesterone, drospirenone, 6β, 7β;15β, 16β-dimethylen-3-oxo-17-pregna-4,9(11)-dien-21, 17β-carbolactone (=9,11-dehydro-drospirenon=WO 2009/146811), 3-beta-hydroxydesogestrel, 3-ketodesogestrel (=etonogestrel), 17-deacetylnorgestimate, 19-norprogesterone, acetoxypregnenolone, allylestrenol, amgestone, chlormadinone, cyproterone, demegestone, desogestrel, dienogest, dihydrogesterone, dimethisterone, ethisterone, ethynodiol diacetate, flurogestone acetate, gastrinone, gestodene, gestrinone, hydroxymethylprogesterone, hydroxyprogesterone, lynestrenol (=lynoestrenol), mecirogestone, medroxyprogesterone, megestrol, melengestrol, nomegestrol, norethindrone (=norethisterone), norethynodrel, norgestrel (including d-norgestrel and dl-norgestrel), norgestrienone, normethisterone, progesterone, quingestanol, (17alpha)-17-hydroxy-11-methylen-19-norpregna-4,15-dien-20-yn-3-one, tibolone, trimegestone, algestone acetophenide, nestorone, promegestone, 17-hydroxyprogesterone esters, 19-nor-17hydroxyprogesterone, 17alpha-ethinyl-testosterone, 17alpha-ethinyl-19-nor-testosterone, d-17beta-acetoxy-13beta-ethyl-17alpha-ethinyl-gon-4-en-3-one oxime or the compounds disclosed in WO 00/66570, in particular tanaproget. Levonorgestrel, norgestimate, norethisterone, drospirenone, dienogest and dydrogesterone are preferred. Drospirenone is particularly preferred.

As estrogens in the medicament according to the invention or in the pharmaceutical combination preparation according to the invention, ethinylestradiol, mestranol, quinestranol, estradiol, esters of estradiol, in particular the valerate or benzoate thereof, estrone, estrane, estriol, estetrol and conjugated equine estrogens are possible. Here ethinylestradiol, estradiol and estradiol valerate are preferred, and ethinylestradiol is particularly preferred.

The quantities of the particular gestagens and/or estrogens correspond to the quantities usually known in oral contraceptives or in oral HT preparations.

For example for the gestagens mentioned below these are normally as follows:

| | | |
|---|---|---|
| Drospirenone | 0.5-5 | mg |
| Levonorgestrel | 30-250 | μg |
| Norgestimate | 180-250 | μg |
| Norethisterone acetate | 0.5-1 | mg |
| Cyproterone acetate | 1-2 | mg |
| Desogestrel | 20-150 | μg |
| Dienogest | 1-3 | mg |
| Gestodene | 60-75 | μg |
| Tibolone | 2.5 | mg |

According to the present invention, the daily administered preferred quantity of drospirenone is 0.5 to 5 mg. In one oral contraceptive (Yasmin®, YAZ®), 3 mg are contained per dosage unit. For the oral HT preparation Angeliq®, modifications with different quantities of drospirenone, for example with 1 or 2 mg of drospirenone, have been developed.

For the estrogens mentioned below, the quantity of estrogen used according to the invention is about:

| | | |
|---|---|---|
| Ethinylestradiol | 10-50 | μg |
| Estradiol | 1-4 | mg |
| Estradiol valerate | 1-4 | mg |
| Mestranol | 50 | μg |

According to the present invention, the preferred daily administered quantity in an oral contraceptive for example based on ethinylestradiol is 10 to 50 μg, particularly preferably 10 to 30 μg, quite particularly preferably 20 to 30 μg.

Oral HT preparations usually contain between 1 and 2 mg of estradiol.

Probiotic bacterial strain is understood to mean either a single bacterial strain or also a combination of several such strains.

As examples of probiotic bacterial strains to be used according to the invention in the medicament according to the invention or in the pharmaceutical combination preparation according to the invention, the following may be mentioned:

Bifidobacterium strains, lactobacillus species, such as for example Lactobacillus reuteri, Lactobacillus reuterii RC-14, Lactobacillus delbrueckii, Lactobacillus gasseri, Lactobacillus jensenii, Lactobacillus catenaforme, Lactobacillus paracasei, Lactobacillus paracasei Lbp PB01, Lactobacillus casei, Lactobacillus acidophilus, Lactobacillus acidophilus Lba EB01, Lactobacillus acidophilus Lba EB02, Lactobacillus crispatus, Lactobacillus crispatus CTV05, Lactobacillus salivarius, Lactobacillus brevis, Lactobacillus fermentum, Lactobacillus fermentum RC-14, Lactobacillus fermentum B-54, Lactobacillus plantarum, Lactobacillus plantarum Lbpl PB02, Lactobacillus Lbxx EB03, Lactobacillus Lbxx PB03, Lactobacillus rhamnosus, Lactobacillus rhamnosus GR-1 and other genera or bacterial strains with essentially the same properties.

Preferably the probiotic bacterial strain is Lactobacillus reuteri, Lactobacillus gasseri, Lactobacillus crispatus and Lactobacillus rhamnosus or a combination of these preferred strains or a combination of at least one of these strains with at least one other of the strains from the above list.

The daily dosage of probiotic bacterial strain is 10⁷ to 10¹¹ CFU (colony forming units), and the daily dosage is preferably 10⁷ to 10⁹ CFU.

### FORMULATION

The person skilled in the art is familiar with the fact that in the production of medicaments containing lactobacillus strains which belong to the Lactobacteriaceae family and as obligate anaerobes excrete lactic acid, limitations have to be considered: thus lactobacillus preparations such as for example dry powders from Lactobacillus acidophilus, which is mostly obtained from a fermentation process by cell concentration followed by freeze-drying, are particularly sensitive on the one hand to moisture and elevated temperature and on the other hand to mechanical stress. On the other hand, for medicaments containing hormone active substances, in many cases production processes such as wet granulation, tabletting and film-coating from aqueous film suspensions are used, so that tablets or film-coated tablets are obtained as drug forms. However, during wet granulation and film-coating, the medicament to be produced is exposed to a moist, warm environment, and during tabletting the formulation components are compressed by the application of high pressures. Owing to the aforesaid sensitivity of lactobacillus preparations, it is therefore not surprising that medicaments for oral use containing lactobacillus are predominantly marketed as non-compressed or only slightly compressed drug forms, for example as apportioned powders or in capsules or in the form of only slightly compressed chewable tablets.

Formulations for use according to the present invention are therefore preferably produced in a manner wherein firstly production processes suitable for the lactobacillus preparations and for the hormones are each used separately from one another and then the lactobacillus preparation and the hormone preparation are combined in one medicament.

Suitable production processes for lactobacillus preparations are known to the person skilled in the art. These in many cases comprise a fermentation process for cell production, cell concentration by centrifugation or separation and a drying process by lyophilization with the addition of several pharmaceutical additives. As suitable additives for the freeze-drying, for example sucrose, microcrystalline cellulose, mannitol, calcium carbonate, magnesium stearate or high disperse silicon dioxide can be used. After completion of the milling process, the dry powder of Lactobacillus acidophilus obtained is for example mixed with one or more pharmaceutical additives. As suitable additives, for example lactose, sucrose, microcrystalline cellulose, mannitol, calcium carbonate, magnesium stearate, high disperse silicon dioxide, antioxidants, vitamins and trace elements may be mentioned (WO 2005060937; EP 00931543; WO 2000195918).

Suitable production processes for hormone preparations (oral contraceptives or HT preparations) are very well known to the person skilled in the art.

The combination of lactobacillus preparations and hormone preparations is for example effected by filling into capsules. For this, for example hard gelatin capsules are opened, and each filled first with a hormone-containing tablet or film-coated tablet and then each filled with a defined volume of a lactobacillus preparation in powder form and then sealed.

Example concerning cell count stability from the production of a probiotic for an oral dosage form:

| | | |
|---|---|---|
| Cell type | Lactobacillus acidophilus | |
| Preparation | Lactobacillus powder | 5 × 10¹⁰ CFU/g |
| Drug form | Capsule (oral) | 5 × 10⁹ CFU/capsule |
| Stability after one year | Room temperature | 5 × 10⁷ CFU/capsule |
| storage at | 2-8° C. | 5 × 10⁸ CFU/capsule |

In women treated with a medicament according to the invention or with a pharmaceutical combination preparation according to the invention, compared to untreated women, i.e. women who received only an oral contraceptive or oral HT preparation with no probiotic bacterial strain, a stabilization of the vaginal environment and associated therewith a lower incidence of the urogenital tract infections described was observed during the treatment and for a week further after the end of the treatment.

### Literature

Allsworth J. E. et al.: Prevalence of Bacterial Vaginosis. Obstetrics & Gynecology, Vol. 109, No. 1, 114-120, January 2007
Anukam K C, et al: Clinical study comparing probiotic Lactobacillus GR-1 and RC-14 with metronidazole vaginal gel to treat symptomatic bacterial vaginosis. Microbes-Infec (8, No. 12-13, 2772-6, 2006)
Chan R. C. Y. et al.: Adherence of Cervical, vaginal and Distal Urethral Normal Microbial Flora . . . Journal of Urology. 1984, Vol. 131, March, 596-601
Cribby S., et al: vaginal Microbiota and the Use of Probiotics. Interdisciplinary Perspectives on Infectious Diseases Volume 2008, Article ID 256490
Specialist information Gynoflor®, Stand 2007
Falagas M E, et al: Probiotics for prevention of recurrent urinary tract infections in women: A review of evidence from microbiological and clinical studies. Drugs 2006, Vol/Iss/Pg. 66/9 (1253-1261), ISSN: 0012-6667
Hay P.: Recurrent Bacterial Vaginosis. Curr Opin Infect Dis 22: 82-86, 2009
Johannsen E, et al: urogenital infections and probiotics. South African Journal of Obstretics and Gynecology 2004, Vol/Iss/Pg. 10/3 (69-71), ISSN: 0038-2329
Kirjavainen P, et al: Expression of anti-microbial defence factors in vaginal mucosa following exposure to Lactobacillus rhamnosus GR-1. not published 2008
Marelli G, et al: Lactobacilli for prevention of urogenital infections: a review. European review for medical and pharmacological sciences, March-April 2004, vol. 8, no. 2, p. 87-95, 118 refs, ISSN: 1128-3602
May A D, et al: Colonization of the rectum by Lactobacillus species and decreased risk of bacterial vaginosis. The Journal of infectious diseases, 1 Aug. 2005, vol. 192, no. 3, p.394-8, ISSN: 0022-1899
Melis G B, et al: Role of pH as a regulator of vaginal physiological environment. Minerva Ginecol 52 (4) (111-21) (2000)
Milsom I, et al: Rational prescribing for postmenopausal urogenital complaints. DRUGS AGING 9 (2) 78-86 (1996)1996
Morelli L, et al: Utilization of the intestinal tract as a delivery system for urogenital probiotics. J Clin Gastroenterol 2004; 38(6): 107-10
Nelson D. B.: Preterm Labor and bacterial vaginosis-associated bacteria among urban women. J. Perinat. Med. 37 (2009) 130-134
Petricevic L et al: Randomized, double-blind, placebo-controlled study of oral lactobacilli to improve the vaginal flora of postmenopausal women. European Journal of Obstetrics & Gynecology and Reproductive Biology 141 (2008) 54-57
Reid G et al a): urogenital infections in women: can probiotic help? Postgraduate medical journal, August 2003, vol. 79, no. 934, p. 428-32, 40 refs, ISSN: 0032-5473 b): Oral use of Lactobacillus rhamnosus GR-1 and Lactobacillus fermentum GR-14 significantly alters vaginal flora: randomized . . . FEMS Immunol Med Microbiol 2003; 35: 131-4
Silva C, et al: Effects of estrogen administration on the colonization capability of lactobacilli and Escherichia coli in the urinary tracts of mice. Methods Mol Biol 268
Simhan H. N. et al: First-trimester cervical inflammatory milieu and subsequent early preterm birth. American Journal of Obstetrics and Gynecology, 2009, 299:377.e1-377.e4
Sobel J. D.: Vulvovaginal candidosis. Lancet 2004; 369: 1961-71

1. A medicament for oral administration containing at least one estrogen and/or one gestagen and at least one probiotic bacterial strain.

2. The medicament as claimed in claim 1, characterized in that the estrogen is selected from the group of the compounds of ethinylestradiol, mestranol, quinestranol, estradiol, and esters of estradiol, in particular the valerate or benzoate thereof, estrone, estrane, estriol, estetrol and conjugated equine estrogens.

3. The medicament as claimed in claim 1, characterized in that the gestagen is selected from the group of the compounds of levonorgestrel, norgestimate, norethisterone, dydrogesterone, drospirenone, 6β,7β;15β,16β-dimethylen-3-oxo-17-pregna-4,9(11)-dien-21,17β-carbolactone, 3beta-hydroxydesogestrel, 3-keto-desogestrel, 17-deacetylnorgestimate, 19-norprogesterone, acetoxy-pregnenolone, allylestrenol, amgestone, chlormadinone, cyproterone, demegestone, desogestrel, dienogest, dihydrogesterone, dimethisterone, ethisterone, ethynodiol diacetate, flurogestone acetate, gastrinone, gestodene, gestrinone, hydroxymethylprogesterone, hydroxy-progesterone, lynestrenol, mecirogestone, medroxyprogesterone, megestrol, melengestrol, nomegestrol, norethindrone, norethynodrel, norgestrel (including d-norgestrel and dl-norgestrel), norgestrienone, normethisterone, progesterone, quingestanol, (17alpha)-17-hydroxy-11-methylen-19-norpregna-4,15-dien-20-yn-3-one, tibolone, trimegestone, algestone acetophenide, nestorone, promegestone, 17-hydroxyprogesterone esters, 19-nor-17hydroxyprogesterone, 17alpha-ethinyl-testosterone, 17alpha-ethinyl-19-nor-testosterone, d-17beta-acetoxy-13beta-ethyl-17alpha-ethinyl-gon-4-en-3-one oxime or tanaproget.

4. The medicament as claimed in claim 1, characterized in that it contains several probiotic bacterial strains.

5. The medicament as claimed in claim 4, characterized in that it contains two probiotic bacterial strains.

6. The medicament as claimed in claim 1, characterized in that the probiotic bacterial strain or the probiotic bacterial strains is/are selected from the group of bifidobacterium strains, lactobacillus species, such as for example Lactobacillus reuteri, Lactobacillus reuterii RC-14, Lactobacillus delbrueckii, Lactobacillus gasseri, Lactobacillus jensenii, Lactobacillus catenaforme, Lactobacillus paracasei, Lactobacillus paracasei Lbp PB01, Lactobacillus casei, Lactobacillus acidophilus, Lactobacillus acidophilus Lba EB01, Lactobacillus acidophilus Lba EB02, Lactobacillus crispatus, Lactobacillus crispatus CTV05, Lactobacillus salivarius, Lactobacillus brevis, Lactobacillus fermentum, Lactobacillus fermentum RC-14, Lactobacillus fermentum B-54, Lactobacillus plantarum, Lactobacillus plantarum Lbpl PB02, Lactobacillus Lbxx EB03, Lactobacillus Lbxx PB03, Lactobacillus rhamnosus, Lactobacillus rhamnosus GR-1 and other genera or bacterial strains with essentially the same properties.

7. The medicament as claimed in claim 6, characterized in that at least one of the probiotic bacterial strains is selected from the group of Lactobacillus reuteri, Lactobacillus gasseri, Lacto-bacillus crispatus and Lactobacillus rhamnosus.

8. The medicament as claimed in claim 1, characterized in that the following are contained as the daily dosage of a gestagen

| | | |
|---|---|---|
| Drospirenone | 0.5-5 | mg |
| Levonorgestrel | 30-250 | μg |
| Norgestimate | 180-250 | μg |
| Norethisterone acetate | 0.5-1 | mg |
| Cyproterone acetate | 1-2 | mg |
| Desogestrel | 20-150 | μg |
| Dienogest | 1-3 | mg |
| Gestodene | 60-75 | μg |
| Tibolone | 2.5 | mg |

9. The medicament as claimed in claim 8, characterized in that as the daily dosage 0.5 to 3 mg of drospirenone are contained in an oral contraceptive.

10. The medicament as claimed in claim 8, characterized in that as the daily dosage 0.5 to 2 mg of drospirenone are contained in an oral HT preparation.

11. The medicament as claimed in claim 1, characterized in that the following are contained as the daily dosage of an estrogen

| | | |
|---|---|---|
| Ethinylestradiol | 10-50 | μg |
| Estradiol | 1-4 | mg |
| Estradiol valerate | 1-4 | mg |
| Mestranol | 50 | μg. |

12. The medicament as claimed in claim 11, characterized in that as the daily dosage 10 to 50 μg, or 10 to 30 μg, or 20 to 30 μg of ethinylestradiol are contained in an oral contraceptive.

13. The medicament as claimed in claim 11, characterized in that as the daily dosage 0.5 to 2 mg of estradiol are contained in an oral HT preparation.

14. The medicament as claimed in claim 1, characterized in that as the daily dosage of probiotic bacterial strain 10⁷ to 10¹¹ CFU (colony forming units), preferably 10⁷ to 10⁹ CFU, are contained.

15. A kit containing at least 20 daily dosage units containing a medicament as claimed in claim 1 and at least one daily dosage unit containing at least one probiotic bacterial strain, wherein the number of all dosage units contained in the kit is at least 28 and the dosage units are arranged such that firstly the dosage units containing the medicament as claimed in one of the previous claims and next the dosage units containing only the probiotic bacterial strain are to be taken.

16. The kit as claimed in claim 15 containing 20 to 30 daily dosage units containing a medicament as claimed in claim 1, and 1 to 10 daily dosage units containing a probiotic bacterial strain.

17. The kit as claimed in claim 16 containing 21 to 26 daily dosage units containing a medicament as claimed in claim 1, and 2 to 7 daily dosage units containing a probiotic bacterial strain, wherein the number of all dosage units contained in the kit is 28.

18. The kit as claimed in claim 15 containing 21 to 26 daily dosage units containing a medicament as claimed in claim 1 and 2 to 7 daily dosage units containing a probiotic bacterial strain.

19. The kit as claimed in claim 15 containing 21 daily dosage units containing a medicament as claimed in claim 1 and 7 daily dosage units containing a probiotic bacterial strain.

20. The kit as claimed in claim 15 containing 24 daily dosage units containing a medicament as claimed in claim 1 and 4 daily dosage units containing a probiotic bacterial strain.

21-22. (canceled)
